(19) United States
(12) Patent Application Publication
(10) Pub. No.: US 2026/0363979 A1
(43) Pub. Date: Nov. 19, 2026

(54) Mild Wash Composition For Limbic System Stimulation

(71) Applicant: Conopco, Inc., d/b/a UNILEVER, Hoboken, NJ (US)

(72) Inventors: Kate Ashley Manning, New Fairfield, CT (US); David Waters, Seymour, CT (US); Bivash Ranjan Dasgupta, Cheshire, CT (US)

(73) Assignee: Conopco, Inc., d/b/a UNILEVER, Hoboken, NJ (US)

(21) Appl. No.: 19/209,269

(22) Filed: May 15, 2025

(51) Int. Cl. A61K 8/365; A61K 8/42; A61K 8/44; A61K 8/46; A61K 8/86; A61M 21/00; A61M 21/02; A61Q 13/00; A61Q 19/10

(52) U.S. Cl. CPC A61K 8/365; A61K 8/42; A61K 8/44; A61K 8/466; A61K 8/86; A61M 21/02; A61Q 13/00; A61Q 19/10; A61K 2800/48; A61M 2021/0016; A61M 2021/0077; A61M 2230/10

(57) ABSTRACT

The invention is directed to a mild wash composition suitable to impart limbic system stimulation. The wash composition is substantially free of sulfate-based surfactants and comprises anionic and amphoteric surfactants wherein the anionic surfactant comprises at least 70% by weight of acyl glycinate and acyl isethionate based on total weight of anionic surfactant in the composition. The wash composition is stable when formulated with at least 1.25% by weight fragrance and no more than 10.25% by weight total surfactant.

### FIELD OF THE INVENTION

The present invention is directed to a mild wash composition suitable to impart limbic system stimulation. More particularly, the wash composition is substantially free of sulfate-based surfactants and comprises anionic and amphoteric surfactants wherein the anionic surfactant comprises at least 70% by weight of acyl glycinate and acyl isethionate based on total weight of anionic surfactant in the composition. Such a wash composition is surprisingly stable even when formulated with at least 1.25% by weight fragrance and no more than 10.25% by weight total surfactant. In addition to being stable, the composition has excellent lather and rinsing characteristics, and may further be formulated substantially free of parabens, silicone oils, hydantoins, phthalates, dioxanes, dyes, isothiazolinones, nonionic surfactants or a mixture thereof.

### BACKGROUND OF THE INVENTION

Wash compositions are typically employed to cleanse skin and to reduce excessive shine associated with sebum produced in epithelial cells known as sebocytes. They are also used to cleanse the hair and scalp and to minimize organisms on the hands and face such that washing is viewed as the most effective way to prevent the spread of germs, like bacteria. In fact, experts believe that periodic washing throughout the day can significantly reduce the number of consumers catching colds and other ailments that often require medical intervention.

Since the pandemic, consumers are more wash conscious, and therefore, attracted to wash compositions that not only help reduce the chances of getting sick, but that also are mild and minimize a dry and stripped skin sensorial sensation. Additionally, given the hectic schedules of many consumers, there is high demand for essential consumer products to do much more than what is expected and ordinary. In fact, it is in vogue for consumer products to provide benefits that are not typically germane to the purpose the product was historically developed for.

Fragrances are known for use in consumer products, including laundry detergents, shampoos and body wash compositions. They are commonly added to products to attract consumers and to impart an aroma on substrates, like skin, hair or clothing, they are applied to or contacted with. In ancient times, many cultures used fragrances for a host of purposes not associated with cleansing, such as medicinal, spiritual and ceremonial or ritualistic purposes. More recently, attempts have been made to use aromatherapy, the use of aromas from fragrance oils to promote, for example, vitality of the mind and body, for relieving stress and depression, and to improve sleep quality. Notwithstanding, it is not routine to formulate stable and mild wash compositions having no more than 10.25% by weight total surfactant and at least 1.25% by weight fragrance such as those containing oils for limbic system stimulation and that are intended to enhance consumer hedonic benefits. Furthermore, it is not routine to formulate compositions comprising surfactants that do not negatively impact the longevity and intensity of the fragrances formulated within such compositions.

The present invention, therefore, is directed to a mild wash composition suitable to impart limbic system stimulation. More particularly, the wash composition is substantially free of sulfate-based surfactants and comprises anionic and amphoteric surfactants wherein the anionic surfactant comprises at least 70% by weight of acyl glycinate and acyl isethionate based on total weight of anionic surfactant in the composition. Such a wash composition is surprisingly stable even when formulated with at least 1.25% by weight fragrance and no more than 10.25% by weight total surfactant. In addition to being stable, the composition has excellent lather and rinsing characteristics, and may further be formulated substantially free of parabens, silicone oils, hydantoins, phthalates, dioxanes, dyes, isothiazolinones, nonionic surfactants or a mixture thereof.

### Additional Information

Efforts have been disclosed for making wash compositions. In WO 2013/092309 A1, perfumed body cleansing compositions that are free of sulfate and PEG-containing surfactants are described.

Other efforts have been disclosed for making wash compositions. In U.S. Patent Application Publication No. 2023/0193164 A1, a scent boosting composition comprising both a liquid fragrance composition and a particulate fragrance composition is described.

Even other efforts have been disclosed for making wash compositions. In WO 2008/090396 A1, perfume compositions and wash-off systems are described.

None of the additional information describes a wash composition having surfactant, fragrance and the ability to impart limbic system stimulation as described and claimed herein.

### SUMMARY OF THE INVENTION

In a first aspect, the present invention is directed to a wash composition comprising:
a) from 3.5 to 6.5%, or from 3.8 to 6.2%, or from 4 to 6%, or from 4.2 to 5.8%, or from 4.5 to 5.7% by weight anionic surfactant comprising at least 70%, or from 75 to 100% or from 80 to 98%, or from 85 to 95%, or from 85 to 92%, or from 95 to 99%, or 100% by weight acyl isethionate and acyl glycinate based on total weight of anionic surfactant in the composition, the acyl isethionate to acyl glycinate at a weight ratio from 1:4 to 1:3, or from 1:3 to 1:2, or from 1:2 to 1:1.8, or from 1:1.8 to 1:1.5, or 1:1.5 to 1:1.3;
b) from 2.75 to 5.5%, or from 2.85 to 5.2%, or from 2.9 to 5%, or from 3 to 4.8%, or from 3.2 to 4.2%, or from 3.2 to 4% by weight amphoteric surfactant;
c) at least 1.25%, or from 1.25% to 3.75%, or from 1.35% to 3.2%, or from 1.35% to 2.75%, or from 1.36 to 2.5%, or from 1.37 to 2%, or from 1.4 to 1.8% by weight fragrance;
d) an activator of a peroxisome-proliferator-activated receptor (“PPAR agonist”);
e) thickener comprising a first thickener comprising PEG-150 pentaerythrityl tetrastearate and PPG-2 hydroxyethyl cocamide, and a second thickener comprising a cross-linked acrylate component; and
f) water,
wherein the wash composition does not comprise more than 10.25% by weight total surfactant.

In a second aspect, the present invention is directed to a method for imparting limbic system stimulation for enhancing hedonic benefits of a user of the wash composition of the first aspect of the invention whereby top, middle or base fragrance notes or a combination thereof are released from the composition in packaging, when applied to a surface (e.g., clothing or skin) or both to provide olfactory signals to the user, the olfactory signals triggering or enhancing olfactory modulating brain oscillations detectable by electroencephalography.

In a third aspect, the present invention is directed to a method for imparting limbic system stimulation for enhancing hedonic benefits of a consumer comprising, in no particular order, the steps of:
a. instructing the consumer to hand wash, hair wash, shower, bathe or a combination thereof with the wash composition according to the first aspect of the invention;
b. instructing the consumer to wash, condition or spritz or a combination thereof at least one inanimate object with a composition having fragrance that is at least 90%, and preferably, at least 92%, or from 93 to 98%, or from 94 to 100%, or from 95 to 100%, or from 98 to 100%, or 100% by weight identical to the fragrance used in the wash composition of step (a) based on total weight of the fragrance to produce a fragrance contacted inanimate object; and
c. instructing the consumer to use the fragrance contacted inanimate object of step (b) within a time frame of from immediately to within 120 hours, or within 10 seconds to 72 hours, or within 5 minutes to 48 hours, or within 15 minutes to 36 hours, or within 20 minutes to 24 hours, or within 25 minutes to 12 hours, or within 30 minutes to 6 hours, or within 1 minute to 30 minutes of performing step (a),
   wherein step (b) is performed within a time frame of from immediately to within 2 weeks, or from 1 minute to 1.5 weeks, or from 1.5 minutes to 1 week, or from 1.5 minutes to 5 days, or from 2 minutes to 4 days, or from 2 minutes to 3 days, or from 2 minutes to 2 days, or from 3 minutes to 1 day, or from 3 minutes to 12 hours, or from 3 minutes to 6 hours of step (a).

In a fourth aspect, the present invention is directed to the use of anionic surfactant comprising at least 70% by weight acyl isethionate and acyl glycinate based on total weight of the anionic surfactant, amphoteric surfactant and activator of a peroxisome-proliferator-activated receptor (“PPAR agonist”) in a wash composition to stabilize the wash composition and to maximize limbic system stimulation for enhancing hedonic benefits of a user that smells or detects the fragrance, the wash composition comprising at least 1.25% by weight fragrance, and no more than 10.25% by weight surfactant.

In a fifth embodiment, the present invention is directed to a method for imparting limbic system stimulation for enhancing hedonic benefits of a consumer comprising the steps of:
a. washing skin, hair or both with the wash composition of the first aspect of the invention; and
b. applying to either the skin or hair, after washing, a leave-on composition, examples of the leave-on composition including but not limited to a lotion, balm, ointment, serum, cream, tonic, mousse, sculpting gel, deodorant, antiperspirant, body spray (e.g., spritz, aerosol or perfume), suntan lotion, sunless tanning composition, make-up, make-up remover, micellar water, sanitizer, a combination thereof, or the like,
   the leave-on composition having a fragrance that is at least 90%, and preferably, at least 92%, or from 93 to 98%, or from 94 to 100%, or from 95 to 100%, or from 98 to 100%, or 100% by weight identical to the fragrance used in the wash composition.

In a sixth aspect, the present invention is directed to a method for imparting limbic system stimulation for enhancing hedonic benefits of a consumer comprising, in no particular order, the steps of:
a. washing with the wash composition of the first aspect of the invention; and
b. using an oral care product having fragrance that is at least 90%, and preferably, at least 92%, or from 93 to 98%, or from 94 to 100%, or from 95 to 100%, or from 98 to 100%, or 100% by weight identical to the fragrance used in the wash composition,
   the oral care product being a toothpaste, mouthwash, breath freshener, lozenge or gum.

In a seventh aspect, the present invention is directed to a method for imparting limbic system stimulation for enhancing hedonic benefits of a consumer comprising, in no particular order, the steps of:
a. washing with the wash composition of the first aspect of the invention; and
b. providing in a room an air freshener or candle or spraying the room with an air freshener or a combination thereof having fragrance that is at least 90%, and preferably, at least 92%, or from 93 to 98%, or from 94 to 100%, or from 95 to 100%, or from 98 to 100%, or 100% by weight identical to the fragrance used in the wash composition.

All other aspects of the present invention will more readily become apparent from the description and Examples which follow.

Skin, as used herein, is meant to include skin on the arms (including underarms), face, back, feet, neck, chest, hands, legs, buttocks and scalp. Stable means the wash composition of the present invention surprisingly displays no color change, syneresis, malodor and no flocculate or precipitate formation for at least three (3) weeks, and preferably, for one (1) month at 25 °C, and most preferably, for three (3) months after being stored at 45 °C and as determined by visual inspection from trained panelists. Stable also means that the composition surprisingly does not display a perceptual decrease in fragrance intensity and quality after being stored consistent with the above conditions and as determined by skilled panelists evaluating the composition in an olfactory sensory panel. Viscosity values used herein are measured at 25 °C with a Discovery HR-2 Rheometer using sand blasted plates having a 1 mm gap and a shear rate of 2-4 1/s with equilibrium at 90 sec and with measurements at 30 sec. Lathers and rinses well means having a lather or foam volume and wash off or rinse characteristics consistent with more traditional compositions having no more than 10.25% by weight total surfactant and from 0.5 to 1% by weight fragrance (at least 25% by weight less fragrance) as determined by skilled panelists washing with water at about 37 °C with wash composition and water at a composition to water weight ratio from 1:10 to 1:1.5 or from 1:6 to 1:3 or from 1:5 to 1:4.5 or from 1:4.5 to 1:4, or from 1:4 to 1:2.5.

Isotropic means a composition with micelles arranged without directional preference. The wash composition of the present invention is preferably isotropic. Amphoteric surfactant means a surfactant in a composition that can be positive or cationic at a low or acidic pH, or negative or anionic at a high or basic pH wherein the term amphoteric as used herein is meant to include zwitterionic surfactants, surfactants that can simultaneously maintain both a cationic and anionic charge at a particular pH yet maintain a positive charge at a low or acidic pH. Soap, as used herein, means salt of a fatty acid. Fragrance and fragrance oil as used herein can be used interchangeably and are meant to mean the same whereby a mild wash composition consistent with the invention can have a fragrance with one or more fragrance oils, and therefore, a fragrance used can be a mixture of fragrance oils. The surfactants described herein include salts of the surfactants where counterions are typically sodium, potassium or ammonium ions. Mild surfactant means having synthetic detergent and being substantially free of soap and sulfate-comprising surfactants, including ethoxylated sulfate-comprising surfactants. Inanimate object as used herein means and includes, for example, clothing like pants, slacks, socks, shirts, jackets, and dresses, as well as windows, tabletops, kitchen and bathroom fixtures, tiles, floors, windows, curtains, dishes, utensils, towels, face cloths, sheets, pillow-cases, blankets, quilts, bed spreads, and especially, evening wear like pajamas, night gowns, robes, or the like.

The wash composition of the present invention will typically have a viscosity from 380 mPa-s to 20,000 mPa-s, and preferably, from 750 to 17,500 mPa-s, and most preferably, from 1,000 to 15,000 mPa-s, or from 2,200 to 13,500 mPa-s, or from 2,500 to 12,800 mPa-s, or from 2,800 to 10,000 mPa-s or from 2,900 to 8,100 mPa-s where viscosity is taken via the method previously defined. The wash composition of this invention is suitable to be wiped or washed off, and preferably, washed off with water. Such a composition can be home care cleaning composition (e.g., for tabletops, glass, floors, toilets, dishes, tiles, upholstery, or laundry) but preferably is a shampoo, 2-in-1 wash/conditioner, make-up wash, facial wash, hand wash, or personal care and liquid body wash. In an embodiment of the invention, the wash composition can have a viscosity from 4,200 mPa-s to 8,200 mPa-s. In another embodiment, the wash composition is sold or marketed as a body wash, hand wash, face wash or as a wash for make-up removal. In still another embodiment, the wash composition is sold or marketed as one for use while bathing in the morning or while bathing in the evening.

The wash composition of this invention may be provided as a wash for cosmetic and non-therapeutic uses to remove dirt, oil, make-up or the like from surfaces, especially skin, hair and the scalp where the composition surprisingly imparts excellent limbic system stimulation for a hedonic benefit or experience. In a preferred embodiment, the wash composition is a personal wash composition, and therefore, a body wash for use while showering or bathing. The composition may optionally comprise skin benefit ingredients added thereto such as emollients, vitamins and/or derivatives thereof, resorcinols, retinoic acid precursors, colorants, moisturizers, sunscreens, antibacterial agents, ceramides, mixtures thereof or the like. Such skin benefit ingredients (or agents) can be water soluble, oil soluble or both may be included. If used, oil soluble skin benefit agents typically make up to 1.5% (or up to 0.85%) by weight of the wash composition whereby the activator of a peroxisome-proliferator-activated receptor (“PPAR agonist”) used, such as 12-hydroxystearic acid, is not intended to be included in the weight percent calculation of optional oil soluble skin benefit agent that may be used. Water-soluble skin benefit agents, when optionally used, may make up to 7% (or up to 5%) by weight of the wash composition. The wash composition will typically have a pH from 5.85 to 8.5, and preferably, from 6 to 8, and most preferably, from 6 to 7.8 or from 6 to 7.6, or from 6.1 to 7.5, or from 6.2 to 7.4 or from 6.5 to 7.3. PPAR agonists means compounds that activate receptors identified as peroxisome-proliferator-activated receptors, and especially, a lipid that activates the nuclear receptor PPAR, and particularly, alpha. For the avoidance of doubt, limbic system means brain structures near the thalamus that regulate emotions, motivation and memory, and hedonic benefit (or experience) means benefits derived from enjoying a product, including pleasurable and emotional benefits. As to limbic system stimulation for enhancing or imparting hedonic benefits as described in the invention, such enhancement includes modulating brain oscillations of a consumer (e.g., user of the wash composition) to evoke or influence an emotional state like high activity and action, energy rejuvenation or rebooting, stress reduction and sleep or rest. In an embodiment of the invention, limbic system stimulation for enhancing or imparting hedonic benefits is the result of using product to modulate brain oscillations to influence an emotional state.

As used herein, “substantially free of” as it relates to sulfate-based surfactants including ethoxylated sulfate-based surfactants (e.g., sodium lauryl sulfate or sodium pareth sulfate) means less than 1%, and preferably, less than 0.8%, and most preferably, less than 0.5%, even more preferably, less than 0.2%, or less than 0.1% by weight of the wash composition. In an embodiment of the invention, substantially free of sulfate-based surfactant includes wash composition with 0.0% (i.e., none) by weight sulfate-based surfactant. Regarding the non-sulfate-based ingredients (i.e., ingredients, like a hydantoin preservative, phthalate), substantially free of means less than 0.6% by weight, and preferably, less than 0.4%, and most preferably, less than 0.3%, or less than 0.22%, or less than 0.1%, or less than 0.05% by weight of the wash composition of the invention. As to the non-sulfate-based ingredients, substantially free of also includes 0.0% (i.e., none) by weight of the ingredient in the wash composition of the invention. As to soap (e.g., sodium stearate or sodium oleate), the wash composition can have less than 2%, and preferably, less than 1%, and most preferably, less than 0.75% by weight soap, or less than 0.55%, or less than 0.25%, or from 0.001 to 0.24% or 0.0% (no soap) by weight soap based on total weight of the wash composition. With respect to all ingredients that the wash composition may be substantially free of, it is within the scope of the invention to include from 0.001 to 0.045% by weight of such ingredients. In yet another preferred embodiment, the wash composition of the present invention comprises less than 35 ppm, and preferably, less than 25 ppm, and most preferably, less than 15 ppm dioxane or less than 2 ppm or less than 1 ppm dioxane or 0.0 ppm dioxane based on total weight of dioxane in the wash composition. In another embodiment, the wash composition of this invention may comprise from 0.00001 to 0.00005% by weight dioxane, like 1,4-dioxane or 0.0% by weight 1,4-dioxane.

For the avoidance of doubt, when fragrance is at least 90%, and preferably, at least 92%, or from 93 to 98%, or from 94 to 100%, or from 95 to 100%, or from 98 to 100%, or 100% by weight identical to the fragrance used in the wash composition of the invention (i.e., aqueous based composition) and used in a second product such as laundry detergent or upholstery cleaner, it is within the scope of the invention for such second product to be a bar such as a soap bar or bar free of soap, and that is, 0.0% by weight soap based on total weight of the bar. In an embodiment of the invention, the bars will typically comprise stearic acid, the fragrance, pH buffers, and surfactant, the preferred surfactant including a directly esterified fatty acid isethionate like sodium lauroyl isethionate. In an embodiment of the invention, such bars can comprise starch, cellulose, or both.

The term comprising is meant to encompass the terms consisting essentially of and consisting of. For the avoidance of doubt, and for illustration, the wash composition comprising anionic surfactant, amphoteric surfactant and PPAR agonist is meant to include a composition consisting essentially of the same and a composition consisting of the same. All ranges defined herein are meant to include all ranges subsumed therein. Except in the operating and comparative examples, or where otherwise explicitly indicated, all numbers in this description indicating amounts or ratios of materials or conditions and/or physical properties of materials and/or use are to be understood as modified by the word “about”. The disclosure, as found herein, is to be considered to cover all embodiments as found in the claims as being multiply dependent upon each other irrespective of the fact that claims may be found without multiple dependency or redundancy.

### DETAILED DESCRIPTION OF THE INVENTION

As to the acyl glycinate suitable for use, non-limiting examples include those having the formula:

R(COO)—NHCH₂COO⁻X⁺ I,

where R is a C₅₋₁₉, and preferably, a C₉₋₁₇, and most preferably, a C₁₁₋₁₅, or a C₁₁₋₁₃ alkyl group and X⁺ is a counter ion such as a sodium, potassium or ammonium ion or a mixture thereof. Illustrative examples of the glycinates that may be used in the wash composition include sodium lauroyl glycinate, sodium myristoyl glycinate, sodium cocoyl glycinate, potassium lauroyl glycinate, sodium myristoyl glycinate, potassium cocoyl glycinate or a mixture thereof. In an embodiment of the invention, sodium cocoyl glycinate, sodium lauroyl glycinate or a mixture thereof are the often-preferred acyl glycinates used. In another embodiment, sodium or potassium lauroyl glycinate or both may be used in the wash composition of the invention. In even another embodiment of the invention, sodium lauroyl glycinate is used. In still another embodiment, the acyl portion of the glycinates is preferably saturated but suitable options can include unsaturated hydrophobic chains with typically no more than 2 double bonds, including cumulated, isolated and conjugated double bonds.

The isethionates suitable for use in the wash compositions include alkoxylated isethionates such as those described in Ilardi et al., U.S. Pat. No. 5,393,466, entitled “Fatty Acid Esters of Polyalkoxylated Isethionic Acid.” The isethionate surfactants can include the reaction product of fatty acids esterified with isethionic acid and neutralized with a base like, for example, sodium or potassium hydroxide. The acyl isethionate surfactant can have the general formula:

RC═O(O)—C(V)H—C(Y)H—(OCH₂—CH₂)_{w}—S(═O)₂O⁻X⁺ II,

where R is as previously defined, and w is an integer from 0 to 4 (1 to 4 for the alkoxylated option), V and Y are each independently hydrogen or an alkyl group having 1 to 4 carbons (preferably methyl) and X⁺ is as described a counter ion such as a Na⁺, K⁺, NH₄⁺, a mixture thereof or the like.

It is also within the scope of the invention, when w is 0, for either the carbon alpha or beta to the sulfonate group to have one C₁₋₄ alkyl substitution in place of hydrogen, preferably a C₁₋₃ alkyl substitution, and most preferably, a methyl group. It is further within the scope of the invention for R to have the degree of unsaturation as previously described.

Illustrative examples of the isethionates suitable for use in the wash composition include sodium capryl isethionate, sodium caproylyl isethionate, sodium capryl methyl isethionate, sodium caproylyl methyl isethionate, sodium cocoyl isethionate, sodium cocoyl methyl isethionate, sodium lauroyl isethionate, sodium lauroyl methyl isethionate, potassium lauroyl isethionate, potassium lauroyl methyl isethionate, sodium oleoyl isethionate, sodium oleoyl methyl isethionate, sodium stearoyl isethionate, sodium stearoyl methyl isethionate, sodium myristoyl isethionate, sodium myristoyl methyl isethionate, sodium palmitoyl isethionate, sodium palmitoyl methyl isethionate, ammonium cocoyl isethionate, ammonium cocoyl methyl isethionate, or a mixture thereof. In an embodiment of the invention, isethionate selected for use comprises sodium lauroyl isethionate, sodium cocoyl isethionate or a mixture thereof. In another embodiment, the isethionate selected consists essentially of or consists of sodium lauroyl isethionate, sodium cocoyl isethionate or a mixture thereof. In an often-desired embodiment, the isethionate is sodium lauroyl isethionate.

As described, the wash composition comprises from 3.5 to 6.5%, or from 3.8 to 6.2%, or from 4 to 6%, or from 4.2 to 5.8%, or from 4.5 to 5.7% by weight anionic surfactant comprising at least 70%, or from 75 to 100% or from 80 to 98%, or from 85 to 95%, or from 85 to 92%, or from 95 to 99%, or 100% by weight acyl isethionate and acyl glycinate based on total weight of anionic surfactant in the composition, the acyl isethionate to acyl glycinate at a weight ratio from 1:2 to 1:3, or from 1:3 to 1:2, or from 1:2 to 1:1.8, or from 1:1.8 to 1:1.5, or 1:1.5 to 1:1.3.

Optional anionic surfactants that may be included in the wash composition with the isethionates and glycinates described include acyl taurates. Illustrative examples suitable for use include sodium methyl lauroyl taurate, potassium methyl lauroyl taurate, sodium methyl myristoyl taurate, potassium methyl myristoyl taurate, ammonium methyl myristoyl taurate, sodium methyl cocoyl taurate, potassium methyl cocoyl taurate, ammonium methyl cocoyl taurate, sodium methyl oleoyl taurate, potassium methyl oleoyl taurate, ammonium methyl oleoyl taurate, sodium lauroyl taurate, potassium lauroyl taurate, ammonium myristoyl taurate, sodium cocoyl taurate, potassium oleoyl taurate, mixtures thereof or the like. In an embodiment of the invention, if a taurate is selected for optional use the taurate used is preferably sodium methyl lauroyl taurate.

In an embodiment of the invention, less than 20%, or less than 15%, or less than 10% or from 0.01 to 10%, or from 1 to 5%, or from 1 to 3%, or 0.0% by weight of the total weight of anionic surfactant used in the wash composition is a taurate.

Acyl glutamates may also optionally be included as anionic surfactant in the wash composition. Glutamates such as C₈ to C₂₀, and preferably, C₁₀ to C₁₈ and most preferably, C₁₂ to C₁₆ or C₁₂ to C₁₄ acyl glutamates where the acyl portion is preferably saturated are suitable for optional use. As is the case with the glycinates and isethionates described, the hydrophobic portion of the glutamates may be unsaturated with no more than 2 double bonds, including cumulated, isolated and conjugated double bonds. Illustrative yet nonlimiting examples of the glutamates that may be used include sodium capryloyl glutamate, sodium lauroyl glutamate, sodium myristoyl glutamate, sodium cocoyl glutamate, sodium stearoyl glutamate, dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium olivoyl glutamate, sodium palmitoyl glutamate, sodium undecylenoyl glutamate, disodium cocoyl glutamate or a mixture thereof. In an embodiment of the invention and if a glutamate is selected for use, sodium lauroyl glutamate, sodium cocoyl glutamate, sodium stearoyl glutamate or a mixture thereof are often preferred for optional use. In another embodiment, if use of a glutamate is desired, sodium myristoyl glutamate, sodium cocoyl glutamate or a mixture thereof may often be desired.

In an embodiment of the invention, less than 20%, or less than 15%, or less than 10% or from 0.01 to 10%, or from 1 to 5%, or from 1 to 3%, or 0.0% by weight of the total weight of anionic surfactant used in the wash composition is a glutamate.

Still other anionic surfactants suitable to be selected for optional use include alkyl sulfates, alkyl sulfonates, alkyl sulfosuccinates, alkyl ether sulfosuccinates, acyl sarcosinates or a mixture thereof.

As noted herein, the wash composition is, preferably, substantially free of surfactants that are sulfated (e.g., sulfate-based or comprising surfactants, e.g., sodium lauryl sulfate) and including those that are alkoxylated (e.g., sodium lauryl ether sulfate)). In an embodiment of the invention sulfated surfactants may be used, and again, preferably, at an amount of less than 1% by weight of the wash composition. If optionally included, the alkyl sulfates can include C₈-C₂₀ alkyl sulfates and/or alkoxylated versions such as sodium lauryl ether sulfate, sodium pareth ether sulfate or a mixture thereof where the ethoxylated portion (EO) is typically on average 1-16 EO groups, preferably 1-3 EO groups. When selected, sodium lauryl sulfate, sodium lauryl ether sulfate, sodium pareth sulfate, ammonium lauryl sulfate or a mixture thereof are illustrative examples that may be included as an option for use. As noted herein, the wash compositions are preferably substantially free of sulfate-based surfactants, and most preferably, have no (0.0%) by weight sulfate-based surfactant.

As to additional anionics including sulfonates that may be selected for optional use with the isethionates and glycinates described, the same can further include alkyl sulfonates, alkyl glyceryl ether sulfonates or a mixture thereof where the alkyl portion is typically a C₈₋₁₈ or C₁₀₋₁₆ hydrocarbon.

Other anionic surfactants suitable for optional use include succinates like disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or a mixture thereof. For the avoidance of doubt, MIPA and MEA refer to monoisopropanolamine and monoethanolamine, respectively.

Acyl sarcosinates suitable for use as optional anionic surfactant include those having a C₈-C₂₀ or C₁₀-C₁₈ or C₁₂-C₁₈ acyl group. Illustrative examples of the sarcosinates that may optionally be used include sodium lauroyl sarcosinate, sodium cocoyl sarcosinate, sodium stearoyl sarcosinate, or a mixture thereof.

In an embodiment of the invention, at least one anionic surfactant included is sodium lauryl sulfosuccinate, sodium myristoyl sulfosuccinate, sodium cocoyl sulfosuccinate, sodium stearoyl sulfosuccinate, sodium laureth sulfosuccinate, sodium pareth sulfosuccinate, disodium laureth sulfosuccinate, disodium lauryl sulfosuccinate, diethylhexyl sodium sulfosuccinate or a mixture thereof. In one embodiment of the invention, acyl sarcosinate makes up from 0.5 to 10% or from 1 to 8% or from 3 to 6% or 4 to 5% by weight of the total weight of anionic surfactant in the wash composition with sodium cocoyl sarcosinate, sodium lauroyl sarcosinate or a mixture thereof often being preferred if a sarcosinate is included.

In yet another embodiment, anionic surfactant may include sodium methyl 2-sulfolaurate or disodium 2-sulfolaurate or both.

In still another embodiment of the invention, anionic surfactant suitable for optional use includes fatty acid amides of an amino acid, i.e., acyl alaninates. Examples suitable for optional use include triethanolamine cocoylalaninate, sodium cocoylalaninate, triethanolamine lauroylalaninate, sodium lauroylalaninate or a mixture thereof. In yet another embodiment, anionic surfactant classified as an acyl alaninate makes up from 0 to 12%, or from 0.1 to 7%, or from 0.2 to 5% by weight of the anionic surfactant in the wash composition.

Still other anionic surfactants suitable to optionally include in the wash composition of the present invention are alpha olefin sulfonates represented by the structure:

R¹—CH₂—(H)C═C(H)(CH₂)ₜ—(CH₂)—S(═O)₂O⁻Z⁺ III,

where R¹ is a C₃₋₁₅ linear, branched, saturated or unsaturated hydrocarbon. When unsaturated R¹ preferably has 1 or 2, and most preferably, 1 double bond (i.e., each carbon atom of the bond being sp² hybridized) and t is 0 or 1. In an embodiment of the invention, t is 0 and R¹ is C₆ to C₁₄, and preferably, C₈ to C₁₄, and most preferably, C₈ to C₁₂, and even more preferably, C₁₀ to C₁₂. In another embodiment of the invention, at least 65%, and preferably at least 75%, and most preferably, from 80 to 100% or from 90 to 100% or from 95 to 100% or from 82 to 94% or from 82 to 90% or from 78 to 87% by weight of the alpha olefin sulfonate optionally used is one which is a C₁₆ olefin sulfonate based on total weight of alpha olefin sulfonate in the wash composition. In even another embodiment, from 96 to 100% or from 92 to 99% or from 90 to 96% by weight of the alpha olefin sulfonate used is a C₁₄ or C₁₆ olefin sulfonate (or mixture thereof) based on total weight of alpha olefin sulfonate in the wash composition. Z⁺ is a counter ion that typically includes a sodium, ammonium or potassium ion or a mixture thereof. In yet another embodiment, if used, 85 to 100% or 100% by weight of the weight of alpha olefin sulfonate used is a C₁₆ surfactant.

In still another embodiment, no more than 12%, and preferably, no more than 7%, and most preferably, no more than 5% by weight of the total weight of anionic surfactant used is alpha olefin sulfonate when the same is included for use. In still another embodiment, from 0.0001 to 4% or 0.0% by weight of the total weight of anionic surfactant used is alpha olefin sulfonate.

Yet another optional anionic suitable for use includes sodium palmitoyl lactylate, sodium stearoyl lactylate, palmitoyl glycolate, stearoyl lactylate or a mixture thereof. Typically, and if used, these anionic surfactants will make up from 0.001 to 13%, or from 0.1 to 7%, or from 0.3 to 5% by weight of the total weight of anionic surfactant in the wash composition. In an embodiment of the invention, the wash composition of the present invention has 0.0% by weight of a lactylate and glycolate.

Amphoteric surfactants suitable for use can include betaines like lauryl betaine, lauryldimethyl betaine, coco betaine, cocamidopropyl betaine, laurylamidopropyl betaine, a mixture thereof or the like. Even other amphoteric surfactants suitable for use include lauryl hydroxysultaine, cocamidopropyl hydroxysultaine or mixtures thereof. In an embodiment of the invention, the amphoteric surfactants that may be used in the wash composition include alkyl amidopropyl hydroxysultaines including palmityl, stearyl and/or oleyl amidopropyl hydroxysultaine, and most preferably, palmityl amidopropyl hydroxysultaine. Other amphoteric surfactants suitable for use include behenyl betaine, capryl/capramidopropyl betaine, stearyl betaine, myristyl hydroxysultaine or a mixture thereof. In another embodiment of the invention, the amphoteric surfactant used comprises cocamidopropyl betaine, lauryl sulfobetaine, cocodimethyl carboxymethyl betaine, laurylhydroxy sulfobetaine, cocoamidopropylhydroxyl sulfobetaine, lauryl hydroxysultaine, a mixture thereof or the like. Additional amphoteric surfactants that may be used include C₁₀₋₁₈ alkylamidopropylamine N-oxides like laurylamidopropylamine N-oxide, C₁₀₋₁₈ alkyldimethylamine N-oxides like lauryldimethylamine N-oxide, C₁₀₋₁₈ alkylbetaines like laurylbetaine, or mixtures thereof. In yet another embodiment, the amphoteric surfactant used comprises lauryl sulfobetaine, lauryl hydroxysultaine or a mixture thereof.

In still yet another embodiment of the invention, the amphoteric surfactant selected comprises coca betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, or mixtures thereof. Typically, the betaine-derived surfactant is selected from coco betaine, cocamidopropyl betaine, behenyl betaine, capryl/capramidopropyl betaine lauryl betaine or a mixture thereof. Particularly preferred betaines include coco betaine and cocamidopropyl betaine. Cocamidopropyl betaine is most preferred.

Other surfactants suitable for optional use that possess charges impacted by pH include cocamine oxide, lauramine oxide, myristamine oxide, palmitamine oxide, stearamine oxide, oleamine oxide, cocamidopropyl amine oxide, lauryl amidopropyl amine oxide, myristyl amidopropyl amine oxide, palmityl amidopropyl amine oxide, stearyl amidopropyl amine oxide, oleamidopropyl amine oxide or a mixture thereof.

Even other additional optional examples of the amphoteric surfactants suitable for use include sodium lauroamphoacetate, sodium cocoamphoacetate, sodium lauroamphoacetate, sodium cocoamphoacetate, cocamphodipropionate or a mixture thereof. In another embodiment of the invention, the amphoteric surfactant employed is at least 85% or at least 90%, or at least 95%, or 97 to 100%, or 100% by weight betaine, and again, preferably, cocamidopropyl betaine.

In an embodiment of the invention, if amphoteric surfactant includes an amphoacetate like, for example, sodium lauroamphoacetate, the wash composition preferably comprises less than 2.6%, and preferably less than 2.2%, and most preferably, less than 1.9%, or less than 1.5%, or less than 1%, or less than 0.6%, or 0.0001 to 0.5% by weight of the amphoacetate. In even another embodiment, no (0.0% by weight) amphoacetate is included as amphoteric surfactant in the wash composition.

Total amphoteric surfactant will typically make up from 2.75 to 5.5%, or from 2.85 to 5.2%, or from 2.9 to 5%, or from 3 to 4.8%, or from 3.2 to 4.2%, or from 3.2 to 4% by weight of the wash composition.

In still another embodiment of the invention anionic to amphoteric surfactant in the wash composition are present at a weight ratio from 3:1 to 2.5:1, or from 2.4:1 to 2.2:1, or from 2:1 to 1.9:1, or from 1.8:1 to 1.7:1, or from 1.7:1 to 1.6:1. In a preferred embodiment, the weight ratio of anionic to amphoteric surfactant is from 2.2:1 to 1.65:1, or from 1.65:1 to 1.55:1. In even another embodiment of the invention, the wash composition comprises from 40 to 75%, or from 45 to 70%, or from 48 to 68%, or from 55 to 65% more anionic surfactant than amphoteric surfactant based on total weight of anionic and amphoteric surfactant in the wash composition.

Nonionic surfactants may optionally be used in wash composition. If used, nonionic surfactants are typically used at levels from 0.0002 to 1.2%, or from 0.002 to 1%, or from 0.2 to 0.7%, or from 0.3 to 0.5% by weight of the wash composition. The nonionic surfactants which may be used include the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic surfactant compounds are alkyl (C₆-C₂₂) phenol condensates with ethylene oxide, the condensation products of aliphatic (C₈-C₁₈) primary or secondary linear or branched alcohols with ethylene oxide, and products made by condensation of ethylene oxide with the reaction products of propylene oxide and ethylenediamine. Other nonionic surfactants include long chain tertiary amine oxides, long chain tertiary phosphine oxides, dialkyl sulphoxides, or the like. In an embodiment of the invention, nonionic surfactants optionally used can include decyl glucoside, lauryl glucoside, polysorbate 20, polysorbate 80, coco glucoside, caprylyl/capryl glucoside mixtures thereof, or the like. Nonionic surfactants having the formula HOOC(CH₂)ᵢ—CH═CH—(CH₂)ₖ(CH₂CH₂O)_{z}H, where i, and k are each independently 5 to 15; and z is 5 to 50 are suitable for optional use. In another embodiment of the invention, i and k are each independently 6 to 12; and z is 15 to 35.

The nonionic surfactant selected for optional use may also include a sugar amide, such as a polysaccharide amide. Specifically, the surfactant may be one of the lactobionamides described in U.S. Pat. No. 5,389,279 to Au et al., entitled “Compositions Comprising Nonionic Glycolipid Surfactants issued Feb. 14, 1995 or it may be one of the sugar amides described in U.S. Pat. No. 5,009,814 to Kelkenberg, entitled “Use of N-Poly Hydroxyalkyl Fatty Acid Amides as Thickening Agents for Liquid Aqueous Surfactant Systems” issued Apr. 23, 1991. An additional nonionic surfactant suitable for optional use includes C₈₋₁₈ alkyl polyglycosides, nonionics characterized by a saccharide unit and a hydrophobic alkyl chain.

In an embodiment of the invention, the anionic surfactant used is glycerol monostearate, cocamide monoethanolamine (CMEA), or a mixture thereof which may optionally make up from 0.06 to 0.75%, or from 0.1 to 0.65%, or from 0.2 to 0.55% by weight of the wash composition if selected for use. In another embodiment, the wash composition of the present invention will have no (0.0% by weight) nonionic surfactant. In even another embodiment, the wash composition will have less than 0.5%, or less than 0.3%, or from 0.0001 to 0.2%, or no nonionic surfactant (0.0%) classified as alkyl polyglycosides based on total weight of the wash composition. In yet another embodiment, the wash composition of the present invention will have less than 0.4%, or less than 0.35%, or less than 0.3%, or less than 0.2%, or less than 0.1%, or from 0.001 to 0.3%, or no (0.0% by weight) CMEA.

In still another embodiment of the invention, cationic surfactants may optionally be used in the wash composition. One class of optional cationic surfactants includes heterocyclic ammonium salts such as cetyl or stearyl pyridinium chloride, alkyl amidoethyl pyrrylinodium methyl sulfate, and lapyrium chloride.

Tetra alkyl ammonium salts are another class of cationic surfactants suitable for optional use. Examples include cetyl or stearyl trimethyl ammonium chloride or bromide; hydrogenated palm or tallow trimethylammonium halides; behenyl trimethyl ammonium halides or methyl sulfates; decyl isononyl dimethyl ammonium halides; ditallow (or distearyl)dimethyl ammonium halides, and behenyl dimethyl ammonium chloride.

Still other types of cationic surfactants that may optionally be included are the various ethoxylated quaternary amines and ester quats. Examples include PEG-5 stearyl ammonium lactate (e.g., Genamin KSL manufactured by Clariant), PEG-2 coco ammonium chloride, PEG-15 hydrogenated tallow ammonium chloride, PEG 15 stearyl ammonium chloride, dipalmitoyl ethyl methyl ammonium chloride, dipalmitoyl hydroxyethyl methyl sulfate, and strearyl amidopropyl dimethylamine lactate.

Even other useful cationic surfactants suitable for optional use include quaternized hydrolysates of silk, wheat, and keratin proteins, and it is within the scope of the invention to use mixtures of the aforementioned cationic surfactants.

If used, cationic surfactants will make up no more than 0.75% by weight of the wash composition. If present, cationic surfactants will typically make up from 0.001 to 0.5%, and more typically, from 0.01 to 0.3% by weight of the wash composition. In another embodiment, the wash composition of the present invention will have no (0.0% by weight) cationic surfactant.

Conventional humectants may optionally be included in the wash composition of the invention to assist in moisturizing skin. These components are generally polyhydric alcohol type materials (polyols) that include glycerol (i.e., glycerine or glycerin), sorbitol, maltitol, mannitol, xylitol, hydroxypropyl sorbitol, ethoxylated glycerol, propoxylated glycerol and mixtures thereof. Most preferred is glycerin, sorbitol or a mixture thereof. The amount of humectant employed may range anywhere from 0.0 to less than 8% by weight of the total weight of the wash composition. Often, and when used, humectant makes up from 0.1 to 8%, and preferably, from 0.2 to 5.25%, and most preferably, from 0.2 to 5% or from 0.3 to less than 5% or from 0.3 to 2.5% by weight, or from 0.2 to 1.5% by weight, or from 0.4 to 1.1% by weight of the wash composition whereby glycerol is typically most preferred. In an embodiment of the invention, glycerol makes up from 0.4 to 1.4%, or from 0.5 to 1.2%, or from 0.6 to 1% by weight of the wash composition. In even another embodiment, the wash composition comprises less than 1.3% by weight monohydric alcohol, and preferably, less than 1.1%, or from 0.0001 to 0.6% by weight monohydric alcohol like ethanol. In a most desired embodiment, the wash composition comprises no monohydric alcohol like ethanol, i.e., 0.0% by weight.

Water typically makes up from 68 to 92%, and preferably, from 72 to 90%, and most preferably, from 74 to 89%, or from 75 to 89%, or from 78 to 88% or from 79 to 87% by weight of the wash composition.

As to the peroxisome proliferator-activated receptor (PPAR agonist) suitable for use in the wash composition, the wash composition will preferably comprise from 0.01 to 2%, and preferably, from 0.1 to 1%, and most preferably, from 0.12 to 0.7%, or from 0.15 to 0.5%, or from 0.18 to 0.35%, or from 0.19 to 0.3% or from 0.2 to 0.28% by weight of the PPAR agonist. In another embodiment of the invention, the agonist used is at least 65%, and preferably, at least 75%, and most preferably, from 80 to 100% or from 85 to 100% or from 90 to 100% or from 60 to 90% or from 90 to 98% or 100% by weight 12-hydroxystearic acid based on total weight of the PPAR agonist in the wash composition. Other PPAR agonists suitable for use with 12-hydroxystearic acid are, for example, 12-keto stearic acid, 16-keto stearic acid, 10-hydroxystearic acid, petroselinic acid, conjugated linoleic acid, 12-hydroxy-9-cis-octadecanoic acid (ricinoleic acid), palmitoleic acid, stearolic acid or a mixture thereof. In even another embodiment, 12-hydroxystearic acid makes up is 100% of the PPAR agonist used. In another embodiment of the invention, 12-hydroxystearic acid makes up from 0.19 to 0.34%, or from 0.2 to 0.3%, or from 0.2 to 0.28% by weight of the wash composition.

Thickener used in the wash composition comprises a first thickener comprising PEG-150 pentaerythrityl tetrastearate and PPG-2 hydroxyethyl cocamide, and a second thickener comprising a cross-linked acrylate component.

The first thickener comprising PEG-150 pentaerythrityl tetrastearate and PPG-2 hydroxyethyl cocamide includes those sold under the name Versathix™ by Croda, Inc. The second thickener, the crosslinked acrylate component (e.g., polymer), preferably is free of cationic groups where suitable nonlimiting examples that may be used include cross-linked polymers of acrylic acid or methacrylic acid, copolymers of acrylic or methacrylic acid with hydrophobic monomers, copolymers of carboxylic acid-containing monomers and acrylic esters, and cross-linked copolymers of acrylic acid and acrylate esters. Polymers of acrylic acid cross-linked with a polyfunctional component may also be used. Thickeners identified as cross-linked acrylate components that are suitable for use in the present invention include those that are commercially available as Carbopol 980, Carbopol 981, Carbopol Ultrez 10 polymer, Carbopol Ultrez 20 polymer as well as Carbopol Aqua SF-1 polymer, the same marketed by Lubrizol. Additional acrylate copolymers suitable for use include Acrymul AM 123R (Synthron), Aculyn 33A Polymer and Aculyn Excel Polymer (Dow Chemical). Illustrative copolymers comprising carboxylic acid containing monomers and acrylic acid ester monomers that are suitable for use include Carbopol 1342, Pemulen TR-1 and Pemulen TR-2 available from Lubrizol. Generally preferred are those rheology modifiers which are known under the CTFA declaration as Carbomer and Acrylates/C₁₀₋₃₀ Alkyl Acrylate Crosspolymer. Others suitable for use include Aculyn 38 and Aculyn 88, the same also classified as cross-linked acrylate components or polymers used as rheology modifiers and provided by Dow Chemical. Even other cross-linked acrylate components suitable for use include polyacrylamides such as Sepigel® 305 and taurate copolymers such as Simulgel® EG and Aristoflex® AVC, the copolymers being identified by respective INCI nomenclature as Sodium Acrylate/Sodium Acryloyldimethyl Taurate and Acryloyl Dimethyltaurate/Vinyl Pyrrolidone Copolymer. Another thickener suitable for use is an acrylate-based polymer made commercially available by Seppic and sold under the name Simulgel INS100.

Total thickener used is typically from 0.8 to 2.2% by weight of the wash composition, and preferably, from 1 to 2.2%, or from 1.1 to 2.1%, or from 1.2 to 2%, or from 1.2 to 1.9%, or from 1.3 to 1.8%, or from 1.3 to 1.77%, or from 1.4 to 1.72% by weight of the wash composition. In an embodiment of the invention, from 75 to 95%, and preferably, from 76 to 92%, and most preferably, from 77 to 90%, or from 80 to 90%, or from 84 to 90% or from 85 to 90% or from 86 to 89% by weight of the total weight of thickener is second thickener, i.e., thickener comprising cross-linked acrylate component. In an embodiment of the invention, the total thickener used will comprise from 85 to 100%, or from 88 to 100%, or from 90 to 100%, or from 91 to 98%, or from 92 to 97%, or from 93 to 99%, or 100% by weight of thickener herein defined as first and second thickener, i.e., thickener comprising PEG-150 pentaerythrityl tetrastearate and PPG-2 hydroxyethyl cocamide, and thickener comprising a cross-linked acrylate component. For the avoidance of doubt, thickener, as used herein, includes components generally classified as rheology modifiers that thicken fluids like the wash composition of the present invention but that exhibit shear thinning properties as well. In still another embodiment, cross-linked acrylate component makes up from 1.3 to 1.95%, or from 1.35 to 1.85%, or from 1.35 to 1.8%, or from 1.38 to 1.7%, or from 1.4 to 1.65%, or from 1.4 to 1.6%, or from 1.4 to 1.5% by weight of the wash composition. In even yet another embodiment, thickener comprising PEG-150 pentaerythrityl tetrastearate and PPG-2 hydroxyethyl cocamide makes up from 0.13 to 0.3%, or from 0.15 to 0.28%, or from 0.15 to 0.27%, or from 0.17 to 0.24%, or from 0.18 to 0.22% by weight of the wash composition. In even another embodiment, only thickener classified as PEG-150 pentaerythrityl tetrastearate and PPG-2 hydroxyethyl cocamide, and cross-linked acrylate component is used, and for example, only Versathix™ and Carbopol Aqua SF-1 polymer.

In an embodiment of the invention, however, up to 15% of the total weight of thickener used can include optional thickeners not identified or classified as PEG-150 pentaerythrityl tetrastearate and PPG-2 hydroxyethyl cocamide, and thickener comprising a cross-linked acrylate component. If used, optional thickeners can include those generally identified as polysaccharides. Examples include fibers, starches, natural/synthetic gums and cellulosics. Representative of the starches are chemically modified starches such as sodium hydroxypropyl starch phosphate and aluminum starch octenylsuccinate. Tapioca starch may often be selected, as can maltodextrin. Suitable gums include xanthan, sclerotium, pectin, karaya, arabic, agar, guar (including Acacia senegal guar), carrageenan, alginate and combinations thereof. Suitable cellulosics include hydroxypropyl cellulose, hydroxypropyl methylcellulose, ethylcellulose, sodium carboxy methylcellulose (cellulose gum/carboxymethyl cellulose) and cellulose (e.g. cellulose microfibrils, cellulose nanocrystals or microcrystalline cellulose). Sources of cellulose microfibrils include secondary cell wall materials (e.g. wood pulp, cotton), bacterial cellulose, and primary cell wall materials. Preferably and when used the source of primary cell wall material is selected from the parenchymal tissue of fruits, roots, bulbs, tubers, seeds, leaves and combinations thereof; and more preferably, is selected from citrus fruit, tomato fruit, peach fruit, pumpkin fruit, kiwi fruit, apple fruit, mango fruit, sugar beet, beet root, turnip, parsnip, maize, oat, wheat, peas or combinations thereof. Even more preferably if used, cell wall material is selected from citrus fruit, tomato fruit and combinations thereof. An often-preferred source of primary cell wall material is parenchymal tissue from citrus fruit. Citrus fibers, such as those made available by Herbacel® as AQ Plus can also be used as a source for cellulose microfibrils. The cellulose sources can be surface modified by any of the known methods including those described in Colloidal Polymer Science, Kalia et al., “Nanofibrillated cellulose: surface modification and potential applications” (2014), Vol 292, Pages 5-31.

In another embodiment, thickener optionally used can include a cationic polymer. Such cationic polymers that may optionally be included are quaternary nitrogen-containing polysaccharides, preferably quaternary nitrogen-containing cellulose ethers, such as those described in U.S. Pat. Nos. 3,472,840; 3,962,418; 4,663,159, and 5,407,919. Particularly preferred are quaternary nitrogen-containing hydroxyethyl celluloses. Examples of such cationic polymers are salts of hydroxyethyl cellulose reacted with a trimethyl ammonium substituted epoxide such as Polyquaternium-10, made commercially available by Dow® as UCARE™ Polymer JR-125, UCARE Polymer JR-400, UCARE Polymer KF, UCARE Polymer JR-30M, UCARE Polymer LR-400, UCARE Polymer LR-30M, UCARE Polymer LK mixtures thereof or the like.

In even another embodiment, thickener optionally used can include cationic polymers known as hydrophobically-modified cationic conditioning polymers such as those made commercially available also by Dow® under the names SoftCAT™ SL 5, SoftCAT SL 30, SoftCAT SL 60, SoftCAT SL 100, SoftCAT SK-L, SoftCAT SK-M, and SoftCAT SK-H. Illustrative yet optional and additional thickeners suitable use in the wash compositions are cationic polymers referred to as Polyquaternium-7, Polyquaternium-44, Polyquaternium 24 or mixtures thereof.

In an embodiment of the invention, the amount of thickener used that is not identified as the first and second thickener, i.e., optional thickener, can make up from 0.0001 to 15%, or from 0.001 to 10%, or from 0.001 to 8%, or from 0.001 to 5%, or from 0.001 to 3%, or from 0.01 to 2%, or from 0.1 to 1% but weight of the total weight of thickener used in the wash composition. In still another embodiment of the invention, only thickener having the first and second thickener defined herein is used, and therefore, 0.0% by weight (none) of the optional thickeners are used.

As to fragrances used, these include an oil or a combination of oils suitable for use in compositions that may be applied topically to consumers such as those generally regarded as safe for application to the hair or skin (including scalp) of a consumer. Such oil or oils are also suitable for contact with fabrics like clothing, upholstery or the like that also would come into the proximity of a consumer or contact with the skin (including scalp) or hair of a consumer. The fragrances used in the wash composition of the present invention can be a single oil or combination of oils and they are typically a combination of 2 to 40 oils, or from 3 to 38 oils or from 3 to 30, or from 3 to 25, or from 3 to 20, or from 3 to 15, or from 4 to 12, or from 5 to 10 oils or from 2 to 4 oils. Such fragrances, therefore, will typically comprise a combination of different perfume raw materials whereby the perfume raw materials can include oils generally classified as aldehydes, ketones, alcohols, ethers, terpenes, terpenoids, imines, thiols, esters, lactones, amides, lactams or the like or a mixture thereof. The perfume raw materials may be aromatic or nonaromatic, plant or floral derived, where derived includes extracts, including purified extracts. The perfume raw materials suitable for use can also be synthetically made. As to the volatility of the fragrances used, it is within the scope of the invention for the fragrances to have “fast-arriving” volatile top notes, or for longer lasting fragrance impact middle notes or base fragrance notes, or a combination thereof.

In an embodiment of the invention, the fragrance oils selected for use will provide fragrance or aroma and fleeting scents (i.e., sillage) having at least 13%, or at least 15%, or at least 20%, or from 20 to 45%, or from 25 to 40%, or from 25 to 35% top notes (i.e., first impression scents) based on total amount of sillage or scents fleeting the fragrance, with the remainder of the fragrance oils employed providing fleeting scents having middle notes (i.e., heart or core notes) and base notes (i.e., foundation, lingering notes). In another embodiment of the invention and as to the fragrances or fragrance oils selected for use, the middle and base notes that typically diffuse are at a percent ratio from 3:10 to 10:3, or from 2:5 to 5:2, or from 3:5 to 5:3, or from 4:5 to 5:4, or from 1:1 based on the total percent of middle and base notes making up the fleeting scent or sillage that is not first perceived as top notes.

Illustrative examples of the oils or extracts suitable for selection in the fragrances used in the mild wash compositions of this invention include lavender, vanilla, jasmine, eucalyptus, spearmint, orange, lemon, ylang-ylang, lime, bergamot, chamomile, pineapple, grapefruit, blackberry, thyme, cucumber, champagne, rosemary, ginger, sage, mint, basil, limonene, balsamic, frankincense (Boswellia carterii), or a mixture thereof. Synthetic mimics of oils which are animal derived are suitable for use. Animal derived oils are not, however, preferred for use or used.

As to aromatherapy applications, in an embodiment of the invention when improved sleep or when stress or anxiety reduction is desired, fragrance oils that are dominate in jasmine, bergamot, vanilla, chamomile, lavender oil or a combination thereof are desired where dominate means at least 50%, or from 50 to 100%, or from 55 to 98%, or from 55 to 95%, or from 60 to 90% or from 70 to 100%, or from 85 to 100% or 100% by weight of the total oil used to create the fragrance will be made up of such oils.

When the condition of the consumer is “sluggish” or depressed or apathetic, fragrance oils selected include orange, frankincense, pineapple, blackberry, grapefruit, lime, spearmint, lemon, eucalyptus, champagne or mixtures thereof where dominate is as previously defined.

As described, fragrances or fragrance oils for consumer limbic system stimulation are intended to enhance consumer hedonic benefits. The same are unexpectedly formulated within the stable mild wash composition of the present invention that comprises no more than 10.25% by weight total surfactant, or from 6.5 to 10%, or from 6.5 to 9.8%, or from 6.8 to 9.7%, or from 7 to 9.6%, or from 7.2 to 9.5%, or from 7.3 to 9.4%, or from 7.4 to 9.3%, or from 7.5 to 9.2%, or from 7.6 to 9.1%, or from 8.3 to 9.2% by weight total surfactant and when fragrance makes up from at least 1.25%, or from 1.25% to 3.75%, or from 1.35% to 3.2%, or from 1.35% to 2.75%, or from 1.4 to 2.5%, or from 1.4 to 2%, or from 1.4 to 1.8% by weight of the wash composition.

Again, the present wash composition is surprisingly stable, notwithstanding the wash composition has no more than 10.25% by weight surfactant and at least 1.25% by weight fragrance, even when the wash composition is substantially free of sulfate-based surfactants.

In an embodiment of the invention, the wash compositions of the present invention comprise less than 0.4%, and preferably, less than 0.2%, and most preferably, less than 0.1% or 0.0% by weight phthalates like diethyl phthalate. In another embodiment, from 0.0 to 0.1%, or from 0.001 to 0.07%, or from 0.01 to 0.05%, or from 0.02 to 0.04% by weight of fragrance booster or fixative is optionally included based on total weight of the wash composition. Such booster or fixative can include sucrose acetate isobutyrate, benzoin, benzyl salicylate or a mixture thereof. In still another embodiment, it is within the scope of the present invention for fragrance to be encapsulated within microcapsules including those that have a clad which is gelatin-based, made with polyvinyl alcohol, cellulose acetate or the like. If the optional encapsulation of fragrance is desired, typically from 5 to 25%, or from 7 to 20%, or from 10 to 15%, or from 1 to 6% by weight of the total weight of fragrance in the wash composition is encapsulated. In an embodiment of the invention, no fragrance (0.0% by weight) is encapsulated and no booster or fixative (0.0% by weight) is used in the wash composition.

In another embodiment of the invention and regarding the fragrances selected for use, such fragrances can comprise terpinoid and non-terpinoid classified organic compounds in a weight ratio from 20:80 to 80:20, or from 30:70 to 70:30, or from 45:55 to 55:45 based on total weight of terpenoid and non-terpinoid in the fragrances selected for use.

In even another embodiment of the invention, the wash compositions can comprise at least one flower extracts, vitamin D, ergocalciferol, retinol, retinyl palmitate, retinyl propionate, chamomilla recutita (matricaria) flower extract), niacinamide, ascorbic acid, linalool, limonene, hexyl cinnamal, Calendula officinalis flower extract, caffeine, ginseng (adaptogens), citronellol, hydroxycitronellal, cyanocobalamin, ergocalciferol, benzyl salicylate, stearic acid, coumarin, withania somnifera root extract, hyaluronic acid, sodium hyaluronate, vitamin B12, potassium chloride, eucalyptus (cineole, eucalyptol), nerolidol, jasmine bergamot, citrus current, mixtures thereof or the like.

In even yet another embodiment, wash compositions consistent with the invention and desired for a revived mood comprise at least one of magnesium, vitamin B12, nerolidew, limonene, linalool, hydroxycitronellal, ergocalciferol, retinyl palmitate, and cyanocobalamin; and those desired for a bright mood comprise at least one of citrus current, citronellol, hydroxycitronellal, limonene, linalool, ginseng root extract, caffeine, ascorbic acid, cyanocobalamin, and ergocalciferol; and those desired for a calm mood comprise at least one of niacinamide, adaptogens, hyaluronic acid, eucalyptus, limonene, linalool, cyanocobalamin, coumarin, citronellol, hexyl cinnamal, and retinyl palmitate; and those desired for a renewed mood comprise at least one of retinol, retinyl palmitate, vitamin D, botanicals, hexyl cinnamal, linalool, limonene, chamomilla recutita flower extract, niacinamide, Calendula officinalis, ergocalciferol and cyanocobalamin.

Such ingredients, when used, collectively make up from 0.001 to 4.5% by weight of the composition, and preferably, form 0.01 to 3.8%, and most preferably, from 0.1 to 3.2%, or from 0.2 to 2.9%, or from 0.2 to 2.7%, or from 0.2 to 2.5%, or from 0.3 to 2.3%, or from 0.3 to 2.3%, or from 0.3 to 1.9%, or from 0.3 to 1.8%, or from 0.4 to 1.5% or from 0.4 to 1.2% by weight of each wash composition.

Optional water-soluble cosmetic agents suitable for use in the wash composition of the invention are limited only to the extent that they are capable of being used by consumers and soluble in the wash composition at the desired pH ranges described.

Illustrative examples of the skin benefit agents suitable to include are acids, like amino acids, such as arginine, valine or histidine. Additional water-soluble benefit agents suitable for use include thiamine (vitamin B1), riboflavin (vitamin B₂), niacinamide (vitamin B₃), vitamin B₅ (Panthenol), vitamin B₆, folic acid (vitamin B₉), vitamin C, mixtures thereof or the like. Water soluble derivatives of such vitamins may also be employed. For instance, vitamin C derivatives such as ascorbyl tetraisopalmitate, magnesium ascorbyl phosphate and ascorbyl glycoside may be used alone or in combination with each other. Other water-soluble benefit agents suitable for use include 4-ethyl resorcinol, water soluble sunscreens like ensulizole (phenylbenzimidizole sulfonic acid), disodium phenyl dibenzimidazole tetra sulfonate or mixtures thereof. Total amount of optional water-soluble benefit agents (including mixtures thereof) when present in the wash composition may range from 0.001 to 7%, preferably from 0.001 to 5%, and most preferably, from 0.01 to 3.5%, or from 1 to 3.3%, or from 1 to 3% by weight of the wash composition. In an embodiment of the invention, less than 1.5%, or from 0.001 to 1% or 0.01 to 0.8% by weight water soluble benefit agent is used based on total weight of the wash composition. In another embodiment of the invention, from 0.01 to 2%, or from 0.01 to 1.6%, or from 0.01 to 1%, or from 0.01 to 0.5% by weight of lactic acid, salicylic acid, mandelic acid, caffeine or a mixture thereof is included in the wash composition, or 0.0% by weight of the same.

It is also within the scope of the present invention to optionally include oil soluble cosmetic agents (i.e., non-water-soluble agents defined as less than 1.0 g of such agent (solute) can be dissolved in 100 ml of 25 °C water (solvent) at atmospheric pressure). The only limitation with respect to such oil soluble cosmetic agents are that the same are suitable for use in a consumer product.

Illustrative examples of the types of oil soluble cosmetic agents that may optionally be used in the wash composition of the invention include components like sunscreens and including but not limited to ethylhexylmethoxycinnamate, bis-ethyl hexyloxyphenol methoxyphenol triazine, 2-ethylhexyl-2-cyano-3,3-diphenyl-2-propanoic acid, drometrizole trisiloxane, 3,3,5-trimethyl cyclohexyl 2-hydroxybenzoate, 2-ethylhexyl-2hydroxybenzoate or a mixture thereof. Other optional oil soluble cosmetic agents suitable for use include resorcinols like thiamidol (isobutylene thiazolyl resorcinol), 4-hexyl resorcinol, 4-phenylethyl resorcinol, 4-cyclopentyl resorcinol, 4-cyclohexyl resorcinol 4-isopropyl resorcinol or a mixture thereof. Also, 5-substituted resorcinols like 4-cyclohexyl-5-methylbenzene-1,3-diol, 4-isopropyl-5-methylbenzene-1,3-diol, mixtures thereof or the like may be used. The 5-substituted resorcinols, and their synthesis are described in commonly assigned U.S. Published Patent Application No. 2016/0000669A1. In an embodiment of the invention, from 0.001 to 3%, or from 0.01 to 2%, or from 0.1 to 1% by weight thiamidol is used in the wash composition.

Even other oil soluble cosmetic agents suitable for use include omega-3 fatty acids, omega-6 fatty acids, climbazole, farnesol, ursolic acid, myristic acid, geranyl geraniol, oleyl betaine, cocoyl hydroxyethyl imidazoline, vitamin E (and its derivatives including alpha-tocopherol acetate), hexanoyl sphingosine, terpineol, thymol, mixtures thereof or the like.

In an embodiment of the invention, the optional oil soluble cosmetic agent used is a retinoic acid precursor. In one embodiment of the invention, the retinoic acid precursor is retinol, retinal, retinyl propionate, retinyl palmitate, retinyl acetate or a mixture thereof. Retinyl propionate, retinyl palmitate and mixtures thereof are typically preferred when oil soluble cosmetic agent is used.

In another embodiment of the invention, the wash composition comprises at least two of 4-ethyl resorcinol, 4-hexyl resorcinol, thiamidol, stearic acid, 12-hydroxystearic acid, palmitic acid, vitamin B3, or a mixture thereof. In an embodiment of the invention and when used such components can make up from 0.01 to 2.8%, or from 0.1 to 2.3%, or from 0.2 to 2% by weight of the wash composition. In even another embodiment, the wash composition comprises from 0.01 to 1% or from 0.1 to 0.8% by weight thiamidol.

Fatty acids in general are suitable for optional inclusion in the wash compositions of the present invention. These include emollients like stearic acid, palmitic acid or a mixture thereof. Also suitable for use as emollients are C₆-C₁₄ fatty acids, or alcohols and/or amide derivatives thereof. Such components include caproic, caprylic, capric, lauric acid and myristic acid, and any alcohol or amide derivatives of the same may optionally be used.

In an embodiment of the invention, such emollients often make up from 0.0 to 0.7%, or from 0.001 to 1%, or from 0.01 to 0.7%, or from 0.1 to 0.7%, or from 0.2 to 0.4% by weight of the wash composition. In another embodiment of the invention, palmitic acid is used with glycerol and a PPAR agonist, preferably 12-hydroxystearic acid. In another embodiment of the invention, the wash composition comprises less than 0.8%, and preferably, less than 0.5%, and most preferably, less than 0.4%, or less than 0.35%, or less than 0.2%, or no (0.0%) by weight lauric acid. In even another embodiment, lauric acid makes up from 0.0 to 0.35% or from 0.001 to 0.32%, or from 0.01 to 0.28%, or from 0.01 to 0.2%, or from 0.1 to 0.2% by weight of the wash composition.

When optional oil soluble cosmetic agent is used in the wash composition, such cosmetic agent typically makes up to 1.5%, and preferably, from 0.001 to 1.1%, and most preferably, from 0.05 to 1%, or from 0.06 to 0.85% by weight of the wash composition with the proviso that such weight percents as noted exclude the amount of PPAR agonist present in the wash composition.

Conventional preservatives can desirably be incorporated into the wash composition to protect against the growth of potentially harmful microorganisms. Cosmetic chemists are familiar with appropriate preservatives and routinely choose them to satisfy preservative challenge tests and to provide product stability. Suitable traditional preservatives that may be used include those traditionally found in consumer products. Preservatives suitable for preferred use are iodopropynyl butyl carbamate, phenoxyethanol, hydroxyacetophenone, ethylhexylglycerine, imidazolidinyl urea, sodium dehydroacetate, benzyl alcohol, sodium benzoate and mixtures thereof. Other preservatives suitable for use include sodium dehydroacetate, chlorophenesin and decylene glycol. The preservatives should be selected having regard for the use of the composition and possible incompatibilities between the preservatives and other ingredients included. Preservatives are preferably employed in amounts ranging from 0.01% to 2.3%, or 0.1 to 2%, or 0.1 to 1.5% by weight of the total weight of the wash composition. Often preferred is a preservative system with hydroxyacetophenone alone or in a mixture with other preservatives. While suitable for use, but in an embodiment of the invention, the wash composition is substantially free of hydantoins, parabens and isothiazolinones.

In another embodiment, the preservative used is phenoxyethanol, iodopropynyl butyl carbamate or a mixture thereof (present at amounts from 0.2 to 1% or 0.3 to 0.9% or 0.4 to 0.8% by weight of the wash composition). In even another embodiment, traditional emollient like 1,2-octanediol is used with preservative like sodium benzoate, phenoxyethanol, iodopropynyl butyl carbamate or a mixture of the same where the vicinal diol, if used, makes up from 0.5 to 1.5% or from 0.6 to 1.3% or from 0.7 to 1.2% times the amount of preservative used. Lipidated glycines are also suitable for use to boost preservative efficacy. If selected for use, such lipidated component may make up from 0.01 to 1.3% or from 0.1 to 0.9% or from 0.15 to 0.8% by weight of the wash composition. Often preferred lipidated components suitable for use are capryloyl glycine, undecylenoyl glycine or a mixture thereof. In an embodiment of the invention, no (0.0% by weight) vicinal diol and lipidated glycines are used. In another embodiment, from 0.1 to 1.42% by weight lipidated glycine is used as preservative and provided by Galaxy Surfactants, Ltd. Additional preservative combinations include those having phenoxyethanol, benzoic Acid capryloyl glycine and undecylenoyl glycine. In even another embodiment, the composition can comprise from 0.1 to 1.8%, or from 0.2 to 1.5%, or from 0.3 to 1.2%, or from 0.4 to 1% by weight sodium benzoate.

In even another embodiment, the wash composition comprises less than 0.6%, and preferably, less than 0.4%, and most preferably, less than 0.2%, or from 0.001 to 0.3% or no (0.0%) by weight phenoxyethanol. In still another embodiment, the wash composition comprises no (0.0%) by weight iodopropynyl butyl carbamate. In even another embodiment, Galguard LipoG commonly known to include undecylenoyl glycine and capryloyl glycine is selected for use and can in one embodiment make up from 0.1 to 0.7, or from 0.2 to 0.6, or from 0.2 to 0.5% by weight of the wash composition.

In even another embodiment, the wash composition made according to the invention may comprise thymol, terpineol, chloroxylenol, zinc pyrithione, piroctone olamine or a mixture thereof. In still another embodiment, the wash compositions may comprise N-acetyl methionine, N-acetyl cysteine (including D, L isomers of the same) or both. When selected for optional use, such components may make up alone or collectively from 0.01 to 2.5% or from 0.1 to 1.5% or from 0.2 to 1.5% or from 0.1 to 1% or from 0.1 to 0.6% by weight of the wash composition, with the proviso that oil soluble active does not exceed more than 1.5% by weight of the wash composition.

In yet another embodiment, topical antiseptic agents may be added such as those selected from the group consisting of tertrabutylammonium chloride, benzalkonium chloride, benzethonium chloride, cetrimonium chloride, dihydroxypropyltrimonium chloride, cocamidopropyl trimonium chloride, 1-1-dodecanaminium, N,N,N-trimethyl chloride, 1-octadecanaminium, N,N-dimethyl-N-octadecyl chloride or a mixture thereof. If used, such agents can make up from 0.01 to 1%, or from 0.01 to 0.6%, or from 0.02 to 0.5% by weight of the wash composition. In an embodiment of the invention, less than 0.45%, and preferably, less than 0.35%, and most preferably, less than 0.25%, or less than 0.15%, or no (0.0%) by weight of such topical antimicrobial agents. In another embodiment of the invention, less than 0.33%, and preferably, less than 0.23%, and most preferably, less than 0.13% or no (0%) by weight cetrimonium chloride is used in the wash composition. In still another embodiment of the invention, from 0.01 to 0.9%, or from 0.1 to 0.8%, or from 0.2 to 0.6% by weight eugenol, cinnamaldehyde or both are included in the wash composition. In yet another embodiment of the invention, the wash composition comprises thymol, terpineol, and in another embodiment the wash composition comprises no (0.0%) silver salt like Ag₂O and AgSO₄.

Chelators (like EDTA, citric acid, salicylic acid, sodium gluconate, triethanolamine), colorants (like caramel, beet juice, beta-carotene, vegetable juice or mixtures of the same), hydrated silica, alcohol solvents (like polypropylene glycol, PPG-7, PPG-9 or both), and exfoliants (e.g., salt or sugar scrubs) may optionally be included in the wash composition. Each of these substances may range from about 0.03 to about 4%, preferably between 0.01 and 3% (or from 0.05 to 1.2% or 0.07 to 1%) by weight of the total weight of the wash composition. To the extent exfoliants are used, those selected should be of small enough particle size so that they do not impede the performance of any packaging used to dispense the wash composition of the invention. In an embodiment of the invention, less than 0.7%, or less than 0.4%, or from 0.1 to 0.3%, or 0.0% by weight chelator, including EDTA, is present in the composition. In another embodiment, fixative optionally makes up from 0.01 to 0.3%, or from 0.01 to 0.4%, or from 0.1 to 0.2%, or 0.0% by weight of the wash composition.

In still another embodiment of the invention, the wash composition will be substantially free of a sulfate-based surfactant and comprise at least one of thiamidol, oleyl adapalenate, hyaluronic acid, magnolia extract, 10-hydroxystearic acid, 12-hydroxystearic acid, mandelic acid, lactic acid, caffeine, tretinoin ((all-E)-3,7-dimethyl-9-(2,6,6-trimethyl-1 cyclonexen-1-yl)-2,4,6,8-nonatetraenoic acid), epigallocatechin gallate, adapalene (6-[3-(1-adamantyl)-4-methoxyphenyl]naphthalene-2-carboxylic acid), 2-mecaptonicotinoyl glycine, salicylic acid, citric acid, benzoyl peroxide, N-acetyl-DL-methionine, terpineol, thymol, glycerin, retinol, retinyl palmitate, retinyl propionate, 4-ethyl resorcinol, 4-hexyl resorcinol, or a mixture thereof. In another embodiment and when used, such ingredients may make up, collectively, from 0.00001 to 4%, or from 0.001 to 3%, or from 0.01 to 2.5%, or from 0.01 to 2.2%, or from 0.01 to 2%, or 0.1 to 1.5% or 0.1 to 1.25 or 0.1 to 0.8%) by weight of the wash composition whereby oil soluble agent does not make up more than 1.5% by weight of the composition.

In even another embodiment of the invention, the anionic surfactant used is a sodium lauroyl isethionate and sodium lauroyl glycinate and the amphoteric surfactant is cocamidopropyl betaine with no other surfactant (0.0%) by weight additional surfactant included in the wash composition.

In yet another embodiment of the invention, the wash composition comprises at least one of glyceryl oleate, acetylated lanolin alcohol, leaf extract from aloe vera, panthenol, kaolin, ozokerite, cyclodextrin, squalane, squalene, jojoba oil, moringa oil, argan oil, rapeseed oil, sunflower seed oil, almond oil, coffee seed oil, soybean oil, sea-buckthorn oil, hydrogenated soybean oil, shea butter, sunscreen or a mixture thereof. If used, alone or collectively, such ingredients will make up to no more than 1.5% by weight of the wash composition, or from 0.001 to 1.45%, or from 0.01 to 1.35%, or from 0.1 to 1%, or from 0.1 to 0.5% by weight of the wash composition. In an embodiment of the invention, the wash composition can comprise soybean oil and hydrogenated soybean oil at from 0.1 to 1.5% by weight of the wash composition, or from 0.5 to 1.5% or from 0.8 to 1.45% by weight of the composition where soybean oil and hydrogenated soybean oil are present at a weight ratio from 35:65 to 65:35, or from 40:60 to 60:40, or from 45:55 to 55:45, or 50:50. In yet another embodiment, when a mixture of soybean oil and hydrogenated soybean oil are included, from 25 to 50% by weight of oil in the mixture or combination will have oil droplets (in the nanometer size range) from 4 to 550 nm, or from 5 to 450 nm, or from 6 to 275 nm with the remainder of the oil being at a droplet size from 2 to 250 microns, or from 3 to 165 microns, or from 4 to 95 microns, or from 5 to 30 microns where droplet size (volume moment mean diameter D[4,3]) may be obtained following the conventions described in ASTM E 799, Practice for Determining Data Criteria and Processing for Drop Size Analysis. For the avoidance of doubt, D[4,3] is the De Brouckere mean, which is taken by dividing the sum of the 4^{th} power of Di* percentage by the 3^{rd} power of the same calculation.

When making the wash composition, the desired ingredients may be mixed with conventional apparatus under moderate shear and atmospheric conditions, with temperature being from 30 to 85 °C whereby shear continues until a homogeneous product is recovered. When adding fragrance, it is within the scope of the invention to first cool the wash composition and add the fragrance last and when the composition is at a temperature between 22 to 26 °C. The wash composition is suitable to make via a batch or continuous process.

As suggested, the wash composition of this invention may be used at any time, whereby the wash composition comprising fragrance oil that includes orange, frankincense, pineapple, blackberry, grapefruit, lime, spearmint, lemon, eucalyptus, champagne or mixtures thereof is best used when the consumer desires to become more alert, and therefore, used within one to two hours, or within 5 minutes to one hour, or within 10 minutes to 30 minutes of desiring maximum awareness or cognitive advancement.

As also suggested and when sleep or stress and anxiety reduction is desired (i.e., decompressing or unwinding), fragrance oil comprising jasmine, bergamot, vanilla, chamomile, lavender, mint oil or a combination thereof is best used within one to two hours, or within 5 minutes to one hour, or within 10 minutes to 30 minutes of feeling anxious or wanting to relax or even go to sleep.

In an embodiment of the invention, fragrances having floral, white floral and woody notes are desired for a replenishment and awareness recharge, an energetic mood state, i.e., a revived mood. Such fragrances are suitable to have a textured floralcy, dewy green thread blended with creamy textures giving a feeling of replenishment and recharged mood state.

In another embodiment of the invention, fragrances having fruity, tropical fruit and citrus notes are desired for an enjoyable and aware state of mind, a comfortable mood, i.e., a bright mood. Such fragrances are suitable to have fun and experiential, bold fruitiness with an addictive hook and enjoyable transportive scent, yielding the comfortable and bright mood.

In still another embodiment fragrances having floral, aromatic, and amber notes are desired for a sense of focus and a calm mind, relaxed mood state, i.e., a calm mood. Such fragrances are suitable to have comforting textural floral, modern aromatic notes mixed with blurred warmth presenting feelings of rest & mindfulness to yield the calm mood.

In yet another embodiment, floral, white floral and musky notes are desired for an immersive dreamy and relaxed experience, i.e., renewed mood. Such fragrances are suitable to have an intoxicating floralcy with prestige construction, premium complexity and a pronounced hook that gives an immersive dreamy and relaxed experience to yield the renewed mood.

In an embodiment of the invention, mint oil or extract is included in the fragrances used in the invention when imparting a relaxed and calm mood is desired.

The notes described are as determined via olfactory stimulus (i.e., sniffing, the physical smelling of the fragrance by a skilled fragrance expert or perfumer at about 10 to 15 cm from the nose).

As described herein, the invention defined further includes a method for imparting limbic system stimulation for enhancing hedonic benefits of a consumer comprising, in no particular order, the steps of having the consumer hand wash, hair wash, condition, shower, bathe or a combination thereof with the wash composition according to the invention; and having the consumer wash, spritz, or both at least one inanimate object with a composition having fragrance that is at least 90%, and preferably, at least 92%, or from 93 to 98%, or from 94 to 100%, or from 95 to 100%, or from 98 to 100%, or 100% by weight identical to the fragrance used in the wash composition the consumer washes, conditions or bathes with based on total weight of the fragrance used in the composition used to contact the inanimate object; and instructing the consumer to use the fragrance contacted inanimate object immediately to within 72 hours, or within 1 minute to 60 hours, or within 5 minutes to 48 hours, or within 15 minutes to 36 hours, or within 20 minutes to 24 hours, or within 25 minutes to 12 hours, or within 30 minutes to 6 hours, or within 1 minute to 30 minutes of performing the washing step such that the inanimate object is preferably contacted with such fragrance immediately to within 2 weeks, or from 1 minute to 1.5 weeks, or from 1.5 minutes to 1 week, or from 1.5 minutes to 5 days, or from 2 minutes to 4 days, or from 2 minutes to 3 days, or from 2 minutes to 2 days, or from 3 minutes to 1 day, or from 3 minutes to 12 hours, or from 3 minutes to 6 hours of step of when the consumer washes himself or herself.

In an embodiment of the invention the consumer could wash or spritz his or her clothing, including pajamas, bed linens (e.g., sheets, pillowcases, blankets), or any combination thereof utilizing, a laundry detergent, spritz composition or both where the detergent and spritz composition would have fragrance as defined, at least 90% by weight identical to the fragrance used in the wash composition of the invention.

In another embodiment, it is within the scope of the invention to employ art recognized dryer sheets or fabric softeners or both having fragrance as defined, i.e., having at least 90% by weight identical to the fragrance used in the wash composition of the invention whereby the fragrance would be added in fabric softener or impregnated within dryer sheet wovens in conventional amounts and using art recognized and standard techniques.

As to the spritz suitable to deliver fragrance, such compositions are well known and art recognized, typically having cyclodextrin, glycols, sodium citrate, sodium maleate or both, polyamine polymers, water, alcohol, dialkyl sodium sulfosuccinate, preservative and fragrance, the fragrance typically present at 1 to 5.5% by weight of the composition whereby the composition is preferably delivered via a spray bottle.

If in a laundry detergent, any art recognized formula could be used whereby the fragrance used in the wash composition of the present invention would be included in lieu of standard or traditional laundry detergent fragrance. For illustration, such a suitable laundry detergent could be a liquid detergent having water, alcohol, surfactant, processing aids like acrylamide-methacrylamideopropyltrimethyl ammonium chloride copolymer, enzyme like, for example, amylase, as well as bentonite, preservative, colorants, salts, silicones and glycols as processing aids. Other ingredients can include soils suspenders, cleaning aids and dyes for whitening like shading dyes and fragrance, the fragrance typically present at 0.5 to 4.5% by weight of the laundry detergent whereby the composition is traditionally delivered via a cap for measuring liquid volume. Traditional candles may include the fragrances as described herein, the traditional candles having parrafin wax, beeswax, palm wax, synthetic wax or a mixture thereof as well as 3 to 10%, or from 4 to 8%, or from to 5% fragrance. For the avoidance of doubt, the deodorants or antiperspirants that can describe the fragrances described can be sticks, soft solids, roll-ons, gels, and aerosols and include those having both aluminum-based and non-aluminum based antiperspirant actives. In an often-desired embodiment, the fragrances described herein are used in a room spray including an upholstery or clothing spray such as those commonly made with solvents, water, preservative, odor neutralizers and emulsifiers. It is within the scope of the invention for the fragrances described to also be included in an environmentally approved and safe spray or wash applied and used by the consumers on pets such as dogs or cats. As to the sprays, they are typically made with essential oils, surfactants, glycerin and water and include the fragrances described herein for limbic system stimulation to enhance consumer hedonic benefits. In addition to stimulating the limbic system of the consumer, the compositions may be used to impart a mood on the pet, and again, like a cat or dog of the consumer.

The packaging for the wash composition typically is not limited as long as composition can be dispensed. In an embodiment of the invention, wash composition is sold in a pouch, bottle, jar, tube or canister. The packaging preferably is biodegradable and/or is prepared from recycled materials including post-consumer resins. Refillable bottles and paper bottles are also suitable for use as are those that comprise bamboo. Other suitable formats include impregnating the wash composition in nonwovens like those having cotton, polyester or both, or by providing the end use products in water soluble sachets or pods such as those essentially consisting of polyvinyl alcohol.

In an embodiment of the invention, the wash composition can optionally be dispensed from a foam dispensing pump including one having a mesh pore or aperture size from 40 to 375 microns, or from 45 to 365 microns, or from 50 to 345 microns or from 70 to 335 microns, or from 90 to 315 microns, or from 90 to 125 microns.

When using the wash composition of the invention, the composition is often used with water at a composition to water weight ratio from 1:10 to 1:1.5, or from 1:6 to 1:3 or from 1:5 to 1:4.5 or from 1:4.5 to 1:4, contacted with a surface, like skin, and rinsed off with water. For a relaxing experience, for example, one that reduces anxiety and helps with sleep, the consumer would be instructed to wash when rest is desired, rubbing and massaging the wash composition of the invention into a surface like the skin while washing, showering or bathing, taking the time to enjoy the fragrance especially formulated for a hedonic experience or that, for example, is relaxing in nature. In contrast, should an energy or memory boost be desired or a more upbeat hedonic experience be desired, the consumer would be instructed to do the same with a wash composition formulated with fragrance that induces such a result.

As described, the fragrance or fragrance oils for consumer limbic system stimulation, intended to enhance consumer hedonic benefits, are unexpectedly formulated within the stable mild wash composition of the present invention.

The Examples are provided to facilitate an understanding of the invention. The Examples are not intended to limit the scope of the claimed invention.

Assessment of the surprising performance of the wash compositions consistent with the invention was performed in terms of brain or neural oscillation measurements on consumers after using product. Such an assessment was performed by art recognized electroencephalography and measuring electroencephalographic activity in a manner as described in Pivik, R. T., Broughton, R. J., Coppola, R., Davidson, R. J., Fox, N., & Nuwer, M. R. (1993), Guidelines for the recording and quantitative analysis of electroencephalographic activity in research contexts. Psychophysiology, 30 (6), 547-558. Beta waves, i.e., neural oscillations known to have a frequency from 12.5 to 30 Hz were detected as an increase after panelists used product with a defined fragrance and desiring high attention, activeness, and alertness, consistent with an effect defined or marketed as high activity and action, energy rejuvenation or rebooting, or the like. In contrast, alpha waves, neural oscillations that occur between 8 and 12 Hz, were detected as an increase by electroencephalography after panelists used product with a defined fragrance and were desiring a low stress and relaxed or even sleepy state of mind, consistent with an effect defined or marketed as, stress reduction and sleep, relax or rest.

### Example I

Wash compositions were made by combining the ingredients identified in the Tables. Ingredients were combined and mixed under moderate shear via a batch process. Temperature was held steady at about 45 to 70 °C with pressure being atmospheric. Batch mixing continued until homogeneous wash compositions were recovered. The compositions of Table III, made according to the invention, had a viscosity from about 3,000 to 7,000 mPa-s. All compositions had pH values from 6.8 to 7.1. The weight percents identified mean the weight percent of active based on total weight of the wash composition. Fragrances used were especially selected for limbic system stimulation. The notes of the fragrances chosen unexpectedly ensured stable compositions with fragrance profile to impart a particular and defined mood response. In the Examples provided, four fragrances were tested at amounts noted in the Tables: 1. a fragrance emphasizing floral, white floral and woody notes for a replenishment and awareness recharge, i.e., a revived mood; 2. a fragrance emphasizing fruity, tropical fruit and citrus notes for an enjoyable state and relaxed mood, i.e., a bright mood; 3. a fragrance having floral, aromatic, and amber notes for a sense of focus and a calm mind, i.e., a calm mood; and 4. a fragrance having a floral, white floral and musky notes for an immersive dreamy experience, i.e., renewed mood.

TABLE I
WASH COMPOSITION ONE, control

| Ingredient | Weight % |
|---|---|
| Water | Balance |
| First Thickener* | 0.1 |
| Chelator | 0.05 |
| Stearic Acid | 0.25 |
| Lauric Acid | 0.8 |
| Sodium Lauroyl Isethionate | 2.4 |
| Sodium Lauroyl Glycinate | 3.2 |
| Cocamidopropyl Betaine | 3.5 |
| Second Thickener** | 2 |
| Glycerin | 0.9 |
| Glycol Distearate | 1 |
| Phenoxyethanol | 0.6 |
| Idopropynyl Butylcarbamate | 0.01 |
| Sodium Hydroxide | 0.4 |
| Sodium Chloride | 1.5-1.75 |
| Oil, Emotive | 0.1 |
| Moisturizing Extract | 0.0005 |
| ***Fragrance | 1.5 |

*Versathix ™,
**Carbopol Aqua SF- 1
***Fragrance, each fragrance per the notes profile defined above included in four separate washes.

Four wash compositions made with each fragrance and the ingredients as defined in Table I were not made according to the present invention. PPAR agonist was not included, thickener amounts were not used as defined in the invention and the amounts of surfactants were not consistent with the invention.

The wash compositions defined in Table I failed, 1.5 to 1.75% by weight of fragrance in each composition made resulted in compositions that were not usable. The compositions separated, and displayed syneresis within a week of being made, did not maintain a desirable viscosity and fragrance profile required and accepted by consumers or a combination thereof.

TABLE II
WASH COMPOSITION TWO, control

| Ingredient | Weight % |
|---|---|
| Water | Balance |
| Preservative | 1.5 |
| Sodium Lauroyl Isethionate | 1.25 |
| Sodium Methyl Lauroyl Taurate | 1.25 |
| CMEA, nonionic surfactant | 0.4 |
| Lauric Acid | 0.8 |
| Stearic Acid | 0.25 |
| 12-Hydroxystearic Acid | 0.22 |
| Cocamidopropyl Betaine | 6.5 |
| NaOH | 0.15 |
| Glycerin | 2 |
| *Fragrance | 1.5-1.75 |
| Total | 100 |

*Fragrance, each fragrance per the notes profile defined above included in four separate washes.

Four wash compositions made with the ingredients defined in Table II were not made according to the present invention. The surfactants used were not consistent with the invention, and the presence of lauric acid and nonionic surfactant not consistent with the invention resulted in compositions that were not suitable for use.

The wash compositions defined in Table II failed, 1.5 to 1.75% by weight of fragrance in each composition made resulted in compositions that were not usable. The compositions separated, and displayed syneresis within a week of being made, did not maintain a desirable viscosity and fragrance profile required and accepted by consumers or a combination thereof.

TABLE III
WASH COMPOSITIONS, inventive

| Ingredient Description | Active, % by weight |
|---|---|
| Water | Balance |
| First Thickener* | 0.2 |
| Preservative | 1.3 |
| Stearic Acid | 0.3 |
| 12-Hydroxystearic Acid | 0.25 |
| Sodium Lauroyl Isethionate | 2.4 |
| Sodium Lauroyl Glycinate | 3.2 |
| Cocamidopropyl Betaine | 3.5 |
| Glycerin | 0.9 |
| Sodium Chloride | 1 |
| Sodium Hydroxide | 0.5 |
| Second Thickener* | 1.35 |
| Fragrance** | 1.5-1.75 |
| Total | 100 |

*First thickener, Versathix ™ and a second thickener, Carbopol Aqua SF-1.
**Fragrance, each fragrance per the notes profile defined above included in four separate washes.

Stable and superior wash compositions were made with the ingredients identified in Table III. Fragrance levels used were 1.5 and 1.75% by weight, surfactant totals were maintained at levels no higher than 10.25% by weight (7.9% by weight) of the wash compositions and thickener and PPAR was included consistent with the invention described.

Surprisingly, after storage for three months at 45 °C, the wash compositions displayed no syneresis, precipitate or any evidence of grainy crystallization. Further surprisingly, the wash compositions consistent with the invention and that were stored for three months, after olfactory assessment from trained panelists and perfumers, displayed fragrance notes that were crisp and fresh, consistent with newly made products. Fragrances surprisingly remained stable at 1.5 and 1.75% inclusion in the wash compositions having less than 10.25% by weight total surfactant.

The compositions made according to the present invention were used by trained panelists in side-by-side wash comparison tests using conventional and commercially available liquid body washes with less than 1.0% by weight fragrance and fragrance not provided or delegated to impart limbic system stimulation for enhancing hedonic benefits. The water used for lathering was about 37 °C where composition and water were used at a weight ratio of about 1:3.5. Unexpectedly, it was determined that the wash compositions made consistent with the invention lathered and rinsed just as well as the conventional compositions, including a composition with having about 13% by weight total surfactant.

### Example II

### Mood Assessment:

Under confidentiality, participants visited a test center twice over the course of a 4-day period, for 30-minute sessions. Each participant was seated and fitted with a commercially available, Emotiv Epoc X, electroencephalography (EEG) monitoring device to record brain electrical activity, and a Fitbit Sense 2 watch for collecting heart rate variability (HRV) and galvanic skin response (GSR). A GoPro camera was also placed in front of the participants to capture videos of the sessions for monitoring study quality.

Each participant started the study with a 3-minute baseline phase where they were instructed to remain still for the purpose of taking baseline EEG and physiological measures. Afterwards, jars of compositions made consistent with the invention (as described with Table III) were opened and placed on devices posterior to the noses of the participants and one at a time by the EEG researcher. Participants were instructed to sniff each jar for 2 minutes; each event was followed by a one-minute rest period. The test products were subsequently presented in predetermined random orders, testing (EEG measurements) occurred during each 2-minute period. This procedure was repeated with different product randomization (all four washes) for each of the successive sessions conducted.

Consistent with the above description, forty-five female participants, ages 18-35, were exposed to olfactory stimulus (i.e., physically smelled from about a 15 cm distance the four different body wash compositions made consistent with the invention defined herein with Table III) and with 1.5 to 1.75% by weight fragrance (Revive Mood, Bright Mood, Calm Mood and Renew Mood), while EEG, Heart Rate Variability (HRV) and Galvanic Skin Responses (GSR) were recorded. Subsequently, participants rated the products on valence (pleasantness), arousal (stimulating), intensity and familiarity. All brain and physiological responses were calculated over a two-minute exposure period and averaged across the two testing sessions. Electrode locations not directly measured were estimated via art recognized spatial interpolation methods.

### As to the Results Obtained Via EEG:

Alpha waves, neural oscillations that occur between 8 and 12 Hz, a significant main effect of product was revealed for alpha power at electrodes F3, F4, Fp1, Fp2, Fz, P3, P4, and Pz. Bright Mood and Revive Mood led to significantly lower alpha power in comparison to Renew Mood, and Calm Mood as summarized below.

Alpha Power for EEG channels with significant differences

| | Revive Mood | | Bright Mood | | Renew Mood | | Calm Mood | |
|---|---|---|---|---|---|---|---|---|
| Channel | Mean (SD) | | Mean (SD) | | Mean (SD) | | Mean (SD) | |
| F3 | 0.112 | (0.0165) | 0.1133 | (0.0184) | 0.2339 | (0.0611) | 0.2299 | (0.0456) |
| F4 | 0.1108 | (0.0132) | 0.1148 | (0.02) | 0.2274 | (0.0429) | 0.2222 | (0.0317) |
| Fp1 | 0.113 | (0.0174) | 0.1128 | (0.0185) | 0.2245 | (0.0365) | 0.2178 | (0.0222) |
| Fp2 | 0.1134 | (0.0184) | 0.1111 | (0.0134) | 0.2195 | (0.0207) | 0.224 | (0.0336) |
| Fz | 0.1114 | (0.0166) | 0.1093 | (0.0139) | 0.2131 | (0.0077) | 0.2304 | (0.0437) |
| P3 | 0.1107 | (0.0138) | 0.1129 | (0.0192) | 0.2173 | (0.0198) | 0.2166 | (0.0207) |
| P4 | 0.1104 | (0.0165) | 0.1079 | (0.0088) | 0.2273 | (0.0426) | 0.2216 | (0.0307) |
| Pz | 0.1106 | (0.0141) | 0.1082 | (0.006) | 0.2189 | (0.0345) | 0.2168 | (0.0211) |

Beta waves, i.e., neural oscillations known to have a frequency from 12.5 to 30 Hz and more often 16 to 30 Hz, a significant main effect of product revealed Bright Mood and Revive Mood led to significantly increased beta power in comparison to Renew Mood and Calm Mood as summarized below.

Beta Power for EEG channels with significant differences

| | Revive Mood | | Bright Mood | | Renew Mood | | Calm Mood | |
|---|---|---|---|---|---|---|---|---|
| Channel | Mean (SD) | | Mean (SD) | | Mean (SD) | | Mean (SD) | |
| F3 | 0.1014 | (0.0101) | 0.098 | (0.0019) | 0.0671 | (0.0096) | 0.0709 | (0.0139) |
| F4 | 0.1008 | (0.0107) | 0.099 | (0.0094) | 0.0696 | (0.0128) | 0.0673 | (0.007) |
| Fp1 | 0.0996 | (0.0117) | 0.099 | (0.0073) | 0.0688 | (0.0111) | 0.067 | (0.0077) |
| Fp2 | 0.0986 | (0.0042) | 0.1046 | (0.0191) | 0.0709 | (0.0144) | 0.0691 | (0.0123) |
| Fz | 0.0999 | (0.0091) | 0.1035 | (0.0187) | 0.0686 | (0.0103) | 0.0672 | (0.0089) |
| P3 | 0.1011 | (0.0103) | 0.0988 | (0.0062) | 0.0656 | (0.0022) | 0.0716 | (0.0216) |
| P4 | 0.1011 | (0.0141) | 0.1011 | (0.0149) | 0.0681 | (0.0083) | 0.0673 | (0.009) |
| Pz | 0.105 | (0.0199) | 0.1005 | (0.0097) | 0.0711 | (0.0158) | 0.0689 | (0.0129) |
| T6 | 0.0661 | (0.0038) | 0.0707 | (0.0132) | 0.0674 | (0.0084) | 0.0657 | (0.0027) |

The data surprisingly shows that wash compositions of the present invention surprisingly are stable and suitable to impart limbic system stimulation for enhancing hedonic benefits, notwithstanding the compositions comprise at least 1.25% by weight fragrance and less than 10.25% by weight surfactant.

What is claimed is:

1. A wash composition comprising:
a) from 3.5 to 6.5% by weight anionic surfactant comprising at least 70% by weight acyl isethionate and acyl glycinate based on total weight of anionic surfactant in the composition, the acyl isethionate to acyl glycinate at a weight ratio from 1:2 to 1:3;
b) from 2.75 to 5.5% by weight amphoteric surfactant;
c) at least 1.25% by weight fragrance;
d) an activator of a peroxisome-proliferator-activated receptor;
e) thickener comprising a first thickener comprising PEG-150 pentaerythrityl tetrastearate and PPG-2 hydroxyethyl cocamide, and a second thickener comprising a cross-linked acrylate component; and
f) water,
wherein the wash composition does not comprise more than 10.25% by weight total surfactant.

2. The wash composition according to claim 1 wherein the activator of a peroxisome-proliferator-activated receptor comprises 12-hydroxystearic acid.

3. The wash composition according to claim 1 wherein the fragrance comprises at least one oil which is lavender, vanilla, jasmine, eucalyptus, spearmint, orange, lemon, ylang-ylang, lime, bergamot, chamomile, pineapple, grapefruit, blackberry, thyme, cucumber, champagne, rosemary, ginger, sage, mint, basil, limonene, balsamic, frankincense (Boswellia carterii), civet, musk, ambergris or a mixture thereof.

4. The wash composition according to claim 1 wherein the composition has a pH from 5.85 to 8.5 and a viscosity from 380 to 20,000 mPa-s.

5. The wash composition according to claim 1 wherein the composition is substantially free of sulfate-based surfactant.

6. The wash composition according to claim 1 wherein the thickener makes up from 0.8 to 2.2% by weight of the wash composition and the thickener comprises from 85 to 100% by weight of the first and second thickener based on total weight of thickener in the wash composition.

7. The wash composition according to claim 1 wherein the first thickener makes up from 0.13 to 0.3% by weight of the wash composition and the second thickener makes up from 1.3 to 1.95% by weight of the wash composition.

8. The wash composition according to claim 1 wherein the composition further comprises a water-soluble vitamin, fat soluble vitamin, thiamidol, acyl alaninate sunscreen or a mixture thereof.

9. The wash composition according to claim 1 wherein the composition further comprises N-acetyl methionine, N-acetyl cysteine, eugenol, cinnamaldehyde, a sarcosinate, taurate, alaninate or a mixture thereof.

10. The wash composition according to claim 1 wherein the composition further comprises 4-ethyl resorcinol, 4-hexyl resorcinol, niacinamide or a mixture thereof.

11. A method for imparting limbic system stimulation for enhancing hedonic benefits of a user of the wash composition of claim 1 whereby the user washes with the composition and top, middle or base fragrance notes or a combination thereof are released from the composition in packaging, when applied to a surface or both to provide olfactory signals to the user.

12. The method according to claim 11 wherein the surface is a surface of an inanimate object, skin, hair, or a combination thereof.

13. The method according to claim 11 wherein the method further comprises the steps of instructing a consumer to wash with the wash composition and to use an inanimate object that has been contacted with a fragrance that is at least 90% identical to the fragrance in the wash composition.

14. The method according to claim 11 wherein the method further comprises the step of instructing the user to use an air freshener, candle, pet spray, spritz, hair conditioner or a combination thereof having a fragrance that is at least 90% identical to the fragrance in the wash composition.

15. A method for modulating brain oscillations for enhancing an emotional state of a user of the wash composition of claim 1 whereby the user washes with the composition and top, middle or base fragrance notes or a combination thereof are released from the composition in packaging, when applied to a surface or both to provide olfactory signals to the user.

16. The method according claim 14 wherein the method further comprises the step of using electroencephalography to measure beta waves, alpha waves or both of the user.
